(19) United States
(12) Patent Application Publication
(10) Pub. No.: US 2025/0322680 A1
(43) Pub. Date: Oct. 16, 2025

(54) Methods and Systems for Classifying Cell Colony Health

(71) Applicant: Advanced Instruments, LLC, Norwood, MA (US)

(72) Inventors: Anna Rose Davidson, Bournemouth (GB); Pavlos Demetriou, Bournemouth (GB); Amir Hussain, Bournemouth (GB); Yunus Kalkan, Bournemouth (GB)

(21) Appl. No.: 19/094,009

(22) Filed: Mar. 28, 2025

Related U.S. Application Data
(60) Provisional application No. 63/634,393, filed on Apr. 15, 2024.

(51) Int. Cl. G06V 20/69; G06T 5/50; G06T 7/00; G16H 30/40

(52) U.S. Cl. CPC G06V 20/698; G06T 5/50; G06T 7/0012; G06V 20/695; G16H 30/40; G06T 2207/20221; G06T 2207/30024

(57) ABSTRACT

Systems and methods for using machine-learned models to characterize cell colonies are disclosed. In some embodiments, a machine-learned model includes a first model and, optionally, a second model. A first model may be a convolutional neural network for segmenting images. A second model may be a decision-tree-based and/or ensemble model, such as a random forest model, for example for grading cells or one or more cell cultures. Input for a second model may be based on output from a first model. Timepoint may also be used as an input to a first model and/or second model. Multi-frame images may be used as input to a machine-learned model. In some embodiments, each frame of a multi-frame image is input on a different input channel to a machine-learned model. Decisions about whether to continue culturing or not may be made based on characterization made using a machine-learned model.

### CROSS-REFERENCE TO RELATED APPLICATIONS

This application claims the benefit of U.S. Provisional Application No. 63/634,393, filed Apr. 15, 2024, the disclosure of which is hereby incorporated by reference herein in its entirety.

### TECHNICAL FIELD

The present invention relates to methods and systems for characterizing cells and colonies of cells, and more particularly to characterizing cell adhesion.

### BACKGROUND ART

Many commercial processes depend on culturing (e.g., growing) cells, such as stem cells (e.g., induced pluripotent stem cells (iPSCs)). For example, cell line development and drug toxicity testing each involve growing cells. In many cases, it is required to grow, or attempt to grow, many cell colonies or cultures at once to ensure that at least one is sufficient for its intended use. Moreover, many methods of determining sufficient cell growth and/or cell colony health require either destructively interfering with cells, time intensive manual assessment, and/or growing cells for significant periods of time in order to assess the status of growth.

### SUMMARY OF THE EMBODIMENTS

The present disclosure provides systems and methods for using machine-learned models to enable, inter alia, characterization of cells and/or populations of cells (e.g., colonies of cells and/or cell cultures), for example at early timepoints. For example, cells and/or populations of cells may be characterized within two days of beginning cell culturing (e.g., growth). Information output from machine-learned models or produced based on output from a machine-learned model may be used to, for example, determine cell count and/or cell doubling rate and/or characterize cell colonies (e.g., cell colony health). Using machine-learned models allows for earlier determinations to be made than manual assessments (e.g., due to time, accuracy, and/or cost constraints) and/or assessments to be made without destroying cells. Decisions regarding whether to continue to grow (e.g., culture) cells or populations of cells can therefore be made earlier and avoid unnecessary waste of resources, whether money or labor or both.

Machine-learned models disclosed herein are used to characterize (e.g., classify) (e.g., grade) cells and/or populations of cells (e.g., colonies of cells and/or cell cultures). Machine-learned models disclosed herein may use one or more non-fluorescence (e.g., brightfield) images as input, cither alone or in combination with further input such as, for example, timepoint at which the image(s) were acquired, cell information (e.g., cell type), or a combination thereof in order to perform segmentation and/or classification. A machine-learned model may be used to grade one or more cell colonies, for example each in a well of a multi-well plate, for example a machine-learned model may output a grade for one or more cell colonies based on initial input of one or more images. The grade may be an individual grade for each of the one or more cell colonies or a collective grade for the one or more cell colonies (e.g., where the one or more cell colonies are in wells of a common multi-well plate).

In some embodiments, a method (e.g., a computer-implemented method) is directed to characterizing (e.g., classifying) cell (e.g., induced pluripotent stem cell (iPSC)) colonies (e.g., quality and/or character thereof). The method may include receiving, by a processor (e.g., of a computing device), one or more input images [e.g., a single input image (e.g., single multi-frame image)]. One or more cell colonies may be discernable within each of the one or more input images. The method may further include characterizing (e.g., classifying) (e.g., grading) the one or more cell colonies, by the processor, using a machine-learned model using the one or more input images as input to the model.

Systems of the present disclosure include a processor, a memory, and one or more programs, wherein the one or more programs are stored in the memory and are executable by the processor, the one or more programs comprising instructions for implementing at least a portion of a method disclosed herein.

One or more non-transitory computer readable storage media of the present disclosure include one or more programs comprising instructions for implementing at least a portion of a method disclosed herein.

In some embodiments, a machine-learned model uses one or more fluorescence or non-fluorescence (e.g., brightfield) images of one or more cells or one or more populations of cells (e.g., one or more colonies of cells or one or more cell cultures) as input. One or more multi-frame images may be used as input into a machine-learned model. One or more two-frame (2-frame) images, such as one or more 2-frame whole-well brightfield images, and/or one or more three-frame (3-frame) images, such as one or more 3-frame whole-well brightfield images may be used as input to a machine-learned model. A 2-frame image includes two frames from different z-stack image planes, for example below and at, at and above, or above and below a focal plane. A 3-frame image includes three frames from different z-stack image planes, for example below, at, and above a focal plane. Using such 2-frame or 3-frame images may improve performance over single-frame images. For example, markers of cell adhesion and/or colony health may be more apparent when data from multiple image planes are considered and therefore classification may be improved when a model has been trained on such images and uses such images as input. One or more images used for input into a machine-learned model may be a multi-plane whole-well image. One or more images may be greyscale images (e.g., 2-frame or 3-frame greyscale images), for example many brightfield images are greyscale.

Multi-frame images may be used as input to a machine-learned model disclosed herein. The use of multi-frame images may result in improved characterization (e.g., classification). For example, features discernable in an image (e.g., to a human), such as features characteristic of cell colony character (e.g., health and/or morphology), may be difficult to characterize (e.g., classify) using a single frame image whereas multi-frame images may lead to improved classification, for example because features characteristics of cell colony character are more discernable. In some embodiments, for example where a U-Net architecture is used, different image frames are input on different channels. In some embodiments, a multi-frame image is input into a machine-learned model (e.g., a convolution neural network thereof that segments, by semantic or instance segmentation, image(s)) by inputting different frames on different channels. For example, where a conventional U-Net architecture may use different channels for RGB for input images, in some embodiments of the present disclosure, different channels may be used for different frames for input images.

In some embodiments, a machine-learned model for characterizing cells and/or cell colonies includes one or more models. In some embodiments, a machine-learned model includes a first model and a second model. One or more input images may be input for a first model. Input for a second model may be based on output from a first model. Output from a second model may characterize one or more cell colonies. Output from a second model may be a grade (e.g., an A-D or A-C grade). Output from a first model may be a segmentation one or more images (e.g., into a plurality of qualitative classifications). A segmentation may be provided as an image (e.g., mask) and/or as a tabulation of relative fractions of different classes (e.g., percentage of cells or corresponding to each of multiple classes). In some embodiments, a first model is an artificial neural network, for example a convolutional neural network. A first model may have a U-Net architecture, either a conventional U-Net architecture or a modified U-Net architecture. In some embodiments, only a first model is used. In some embodiments, a second model is a decision-tree-based model and/or an ensemble model, for example a random forest model.

Systems and methods disclosed herein enable facile characterization at early timepoints, which can avoid wasting resources on growing or attempting to grow unsuitable cells or cell colonies. In some embodiments, cell colonies are characterized within no more than 14 days (e.g., within 12 days, within 10 days, within 8 days, within 6 days, or within 4 days) (e.g., and after at least 1 day or at least 2 days) of when cell culturing (e.g., growth) began. Such early characterization of cell colonies may conserve resources. Decisions to continue to culture (e.g., grow) and/or discard one or more cells and/or one or more cell colonies may be based on characterization made using a machine-learned model (e.g., based on output from the machine-learned model). Decisions to continue to culture (e.g., grow) and/or discard one or more cells and/or one or more cell colonies may be made on a cell culture by cell culture (e.g., colony by colony) basis (e.g., on a well by well basis) or on a whole well-plate basis (e.g., based on individual and/or collective grades).

Images used as input to a machine-learned model may be of different wells in a multi-well plate. For example, one image (e.g., stitched image) may be used per well. Thus, an image may be a whole-well image. A method may be used to characterize an entire multi-well plate. In some embodiments, images from are input into a machine-learned model together (e.g., sequentially), for example where output from a machine-learned model is a grade of colonies discernable within the images, for example every colony in a plurality of wells of a multi-well plate. For example, in some embodiments, each image is individually segmented with a first model of a machine-learned model and then the input to a second model of the machine-learned model includes each segmentation output; the second model may output a collective grade. In some embodiments, a method is performed for each image separately (e.g., run through a machine-learned model separately). Thus, in some embodiments, one or more colonies for one well of a multi-well plate may be characterized at a time.

Fluorescence or non-fluorescence images used in methods disclosed herein may be stitched images made up of a set of constituent images (e.g., at least 10, at least 100, or at least 1,000 constituent images). Fluorescence or non-fluorescence images used in methods disclosed herein may correspond to one or more whole wells, for example each well in a multi-well plate. Fluorescence or non-fluorescence images used in methods disclosed herein may be of unstained and/or undyed cells. Fluorescence or non-fluorescence images used in methods disclosed herein may be preprocessed. Preprocessing may include normalization and/or binning [e.g., to bin down by a factor (e.g., 2) from a full resolution]. Machine-learned models may be or have been trained using any such fluorescence images and/or non-fluorescence images.

Systems and methods disclosed herein may be used as part of a cell line development process, a clone colony ranking process, or a pluripotency-based assay, for example.

Any two or more of the features described in this specification, including in this summary section, may be combined to form implementations of the disclosure, whether specifically expressly described as a separate combination in this specification or not.

At least part of the methods, systems, and techniques described in this specification may be controlled by executing, on one or more processing devices, instructions that are stored on one or more non-transitory machine-readable storage media. Examples of non-transitory machine-readable storage media include read-only memory, an optical disk drive, memory disk drive, and random access memory. At least part of the methods, systems, and techniques described in this specification may be controlled using a computing system included of one or more processing devices and memory storing instructions that are executable by the one or more processing devices to perform various control operations.

### BRIEF DESCRIPTION OF THE DRAWINGS

The present teachings described herein will be more fully understood from the following description of various illustrative embodiments, when read together with the accompanying drawings. It should be understood that the drawings described below are for illustration purposes only and is not intended to limit the scope of the present teachings in any way. The foregoing features of embodiments will be more readily understood by reference to the following detailed description, taken with reference to the accompanying drawings, in which:

FIG. 1 is a block flow diagram of systems and methods employing machine-learned models, according to illustrative embodiments of the present disclosure;

FIG. 2 is a graphical representation of a qualitative cell colony health classification overlaid over a brightfield image of the cell colony, according to illustrative embodiments of the present disclosure;

FIG. 3 is a graphical representation of a qualitative classification of cells within a colony, according to illustrative embodiments of the present disclosure;

FIG. 4 is a process flow diagram of a method of characterizing (e.g., classifying) cells, according to illustrative embodiments of the present disclosure;

FIG. 5 is a block diagram of an example network environment for use in the methods and systems described herein, according to illustrative embodiments of the present disclosure;

FIG. 6 is a block diagram of an example computing device and an example mobile computing device, for use in illustrative embodiments of the present disclosure; and

FIG. 7 is a block diagram of an exemplary computing system or device that may be used to perform methods, or portions thereof, described herein.

### DETAILED DESCRIPTION OF SPECIFIC EMBODIMENTS

Disclosed herein are systems and methods for characterizing cell colonies using machine-learned models. In some embodiments, a machine-learned model includes a segmentation model. In some embodiments, a machine-learned model includes a grading model for grading cells and/or cell colonies. It has been found that, in certain embodiments, particular network architectures as described herein, in particular using a combination of models, produce better characterizations (e.g., more accurate grades) than single models, specifically for characterizing certain cell colonies and/or classifying cell colonies in certain manners. In some embodiments, a machine-learned model includes a segmentation model and a grading model. For example, in some embodiments, a machine-learned model includes a first model, such as a segmentation model, to produce a mask with multiple classes from an input image, such as a 2-frame image, and a second model, such as a random forest model, that uses ratios of those classes as an input and outputs a grade. The classes may classify areas of images (e.g., pixels) according to cell characteristics (e.g., cell morphology) and/or colony characteristics (e.g., colony morphology). In some embodiments, a machine-learned model includes (i) an artificial neural network, for example that classifies based on classes, at least some of which correspond to cell morphology and/or colony morphology, and/or (ii) a random forest model that produces output [e.g., classifies (e.g., grades)] based on cell morphology and/or colony morphology. For example, the output may be a grade for a colony discernable in an initial input image, for example that was input into an artificial neural network whose output is used for input to the random forest model.

In some embodiments, one or more images of cells, from example of a cell colony, are used as initial input. Each of one or more images may be of a whole-well, for example in a multi-well plate. In some embodiments, a single image (e.g., a single whole-well image) is used as an input (e.g., the only image input) to a machine-learned model. An image, for example a single image, may be a multi-frame image, such as a 2-frame or 3-frame image. An image may be processed before being input into a first model, for example by normalization (e.g., into an 8-bit image) and/or binning down (e.g., by a factor of 2), for example depending on initial resolution of input image(s).

In some embodiments, a machine-learned model includes a first model for classifying (e.g., segmenting) one or more images. A first model may be an artificial neural network, such as, for example, a convolutional neural network. In some embodiments, a first model is a convolutional neural network that uses a U-Net architecture. U-Net architectures are described in Ronneberger, O., et al., “U-Net: Convolutional Networks for Biomedical Image Segmentation,” arXiv:1505.04597, the disclosure of which is hereby incorporated by reference herein in its entirety. A conventional U-Net architecture uses 64 filters at a first convolutional block and doubles at each proceeding block. In some embodiments, fewer filters are used at a first convolutional block. For example, fewer than 32 filters or fewer than 16 filters (e.g., 11 filters) may be used at a first convolutional block and, optionally, doubled at each proceeding block. A conventional U-Net architecture uses 3-channel input (e.g., RGB input). In some embodiments of the present disclosure, a first model uses multi-channel input (e.g., two channel input or three channel input) with each channel corresponding to a frame in a multi-frame image (e.g., a 2-frame or 3-frame image, respectively). For example, a first model may have a U-Net architecture structured to use two input channels with each corresponding to a different frame of a 2-frame image where one or more 2-frame images are used as input to the model. Semantic or instance segmentation may be used. In some embodiments, for example when classifying cell adhesion, semantic segmentation is sufficient without the need for using instance segmentation. Timepoint of an image (e.g., corresponding to current cell growth or cell colony lifetime) (e.g., based on a seeding day for a cell colony) may be used as an additional input to a first model. A first model, such as one having U-Net architecture, may use a cross-entropy loss function, optionally with dice loss.

In some embodiments, a first model outputs a segmentation of an input image. In some embodiments, a first model outputs a segmentation as an image (e.g., mask) and/or as a tabulation of relative fractions of different classes (e.g., based on a percentage of cells or area corresponding to each of multiple classes, e.g., excluding background). In some embodiments, a first model outputs a segmentation using three classes (and background), four classes (and background), five classes (and background), or six classes (and background). For example, when classifying cell colony health, areas (e.g., cells) in an image may be classified as “long-spindle cells,” “thin-flat cells,” “dead” (or “dead-cell”), or “dense-small.” In some embodiments, output of a first model is post-processed, for example to convert output to confidence score(s) (e.g., per pixel per class). For example, a softmax function may be used to post-process output (e.g., to convert raw logits to confidence scores per pixel per class).

In some embodiments, for example some embodiments of characterizing cell colony health, only a first model is used. In some embodiments, for example some embodiments of characterizing (e.g., classifying) cell colonies, output from a first model is used for input into a second model (e.g., after processing). For example, cell colony health may be characterized using two models.

A second model may be used to convert output from a first model into a further output. For example, output of a second model may characterize a cell colony, for example using a grade. Output of a second model may be a qualitative classification (e.g., of a colony), such as a grade. Output of a second model may be a colony morphology class. Output from a first model may be processed before being input into a second model. For example, if a first model outputs a segmentation, the output may be processed into a ratio of classes (e.g., percentage of non-background area corresponding to each class) with the ratio of classes being used as input to a second model. Timepoint of an image (e.g., corresponding to current cell growth or cell colony lifetime) (e.g., based on a seeding day for a cell colony) may be used as an additional input to a second model. A second model may be an ensemble model, such as a random forest model. A second model may be a decision-tree-based model, such as a random forest model.

A second model may be a random forest model that has been trained using a grid search. One or more of the search space definitions present in Table 1 may be used for training a random forest model.

TABLE 1

| Parameter | Value/Rule |
|---|---|
| Number of trees in random forest | [1, 100] (range) |
| Fraction of the original dataset | [0.1, 1] (range) |
| is given to any individual tree | |
| Maximum depth of an individual | {3, 5, 7, None} (set) |
| tree | |
| Minimum number of samples | [2, 6] (range) |
| required to split a node and is | |
| not a pure node (not a node | |
| where a decision is made) | |
| Minimum number of samples | [1, 6] (range) |
| required at each leaf node | |
| (decision node) | |
| Method of selecting samples | Linked with Row 2. If its True then |
| for training each tree | subsample the training dataset based |
| | on the ratio from 2. for each tree to |
| | build the best estimator, if False use |
| | the whole dataset for each tree) |
| | bootstrap = {False, True} (set) |

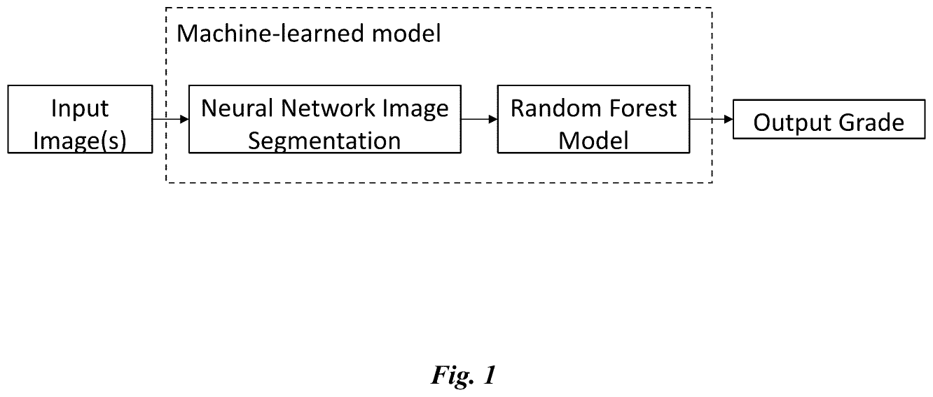

FIG. 1 illustrates exemplary methods of using machine-learned models for classification, according to illustrative embodiments of the present disclosure. In these methods, a machine-learned model used to classify includes an artificial neural network and a random forest model, for example where output from the neural network is used (e.g., after processing) as input to a random forest model. Image(s) are input into the machine-learned model, specifically the neural network image segmentation model of the machine-learned model. The neural network may be a convolutional neural network, for example having a U-Net architecture. In some embodiments, one or more (e.g., a single) multi-frame image is used as the input. Timepoint for the image (e.g., relative to seeding or beginning of culturing (e.g., growth)) may be, but is not necessarily, used as an additional input. The neural network image segmentation model may be structured to receive input on different channels, for example each frame of a multi-frame input may be input on a different channel within the model. The neural network image segmentation model produces output, for example classifying an image (using semantic or instance segmentation) into different classes. Input into the subsequent random forest model of the machine-learned model is based on the output from the neural network image segmentation model, for example either directly or after processing (e.g., converting a segmented image into a ratio of relative class presence within the initial input image(s)). In some embodiments, the artificial neural network may output a segmented image from which a percentage of the image corresponding to different classes (e.g., of cells) may be determined. The random forest classifier outputs a classification, for example, in some embodiments, characterizing cell colony health. The machine-learned model may make classifications based on cell morphology and/or colony morphology. The output of the random forest model is a grade, for example, that characterizes cells and/or one or more colonies.

### Cell Colony Health

During mesenchymal stem cell (MSC) culturing, colony health and pluripotency state are quantified by pluripotency-based assays. Such assays generally include one or more of: a visual grading assessment of colonies, an immunocytochemical based staining, and a functional differentiation protocol. Such approaches are lengthy, unstandardized, subjective and/or destructive.

To address these drawbacks, disclosed herein are methods for classifying cell (e.g., MSC) colonies, for example health of the colonies, using machine-learned models. Methods may include making a classification of a cell colony based on brightfield images, for example label-free brightfield images. In some embodiments, a machine-learned model performs classification based on one or more brightfield images of cells, for example 2-frame (or 3-frame) images such as 2-frame whole-well brightfield images. Images may be stitched images made up of a set of constituent images. Images may be of unstained and/or undyed cells. Images may correspond to one or more whole wells, for example each well in a multi-well plate.

A machine-learned model may be or have been trained on and/or trained to output a number of qualitative cell classifications, for example three or more, four or more, five or more, or six or more classifications. Exemplary classifications that may be used include, for example, “long-spindle cells,” “thin-flat cells,” “dead” (or “dead-cell”), and/or “dense-small.” Output of a machine-learned model may include a qualitative classification for one or more cell colonies. For example, a qualitative classification may be a collective classification for a plurality of colonies, such as, for example, colonies in different wells of a multi-well plate. A qualitative classification may be made based on colony perimeter morphology and/or differentiation of cells in one or more cell colonies and/or timepoint (e.g., since growth of the one or more cell colonies began). A colony classification may be based on, classification of cells within the colony, for example based on an area of the colony corresponding to each cell classification, and/or colony morphology and/or cell type (e.g., percentage and/or number of differentiated and/or undifferentiated cells in the colony). A qualitative colony classification scheme may use an A-C or A-D metric to classify a single colony or one or more colonies collectively (e.g., a set of colonies, such as in different wells of a multi-well plate). In some embodiments, “A” represents a colony including mostly dense small cells, “B” represents a colony including mostly dense small cells and some long spindle cells, and “C” represents a colony including a high amount of dead cells, some long spindle cells, and few dense small cells.

By using a machine-learned model as disclosed herein, earlier classification and/or classification with improved and/or more standardized accuracy of cell colonies may be made, for example at an earlier timepoint. Colony health classification using a machine-learned model may be also include timepoint as an input. For example, one or more cell colonies, or cells therein, discernable in an image (e.g., brightfield image) may be classified differently if the image corresponds to an earlier timepoint or a later timepoint (e.g., with respect to when a cell colony began growing). Cell colony classification may be used to determine whether to continue to grow and/or discard a cell colony or cell colonies, for example within one or more wells of a multi-well plate. Cell colony classification may use a qualitative metric, such as an A-C or A-D metric.

A machine-learned model that classifies cell colonies may be or have been trained using manually annotated brightfield images (e.g., with cells annotated as “long-spindle cells,” “thin-flat cells,” “dead” (or “dead-cell”), and/or “dense-small”). Optionally, fluorescence images may also be used to train a machine-learned model, for example where one or more markers that characterize colony health are fluorescently labelled (and therefore visible in the fluorescence images).

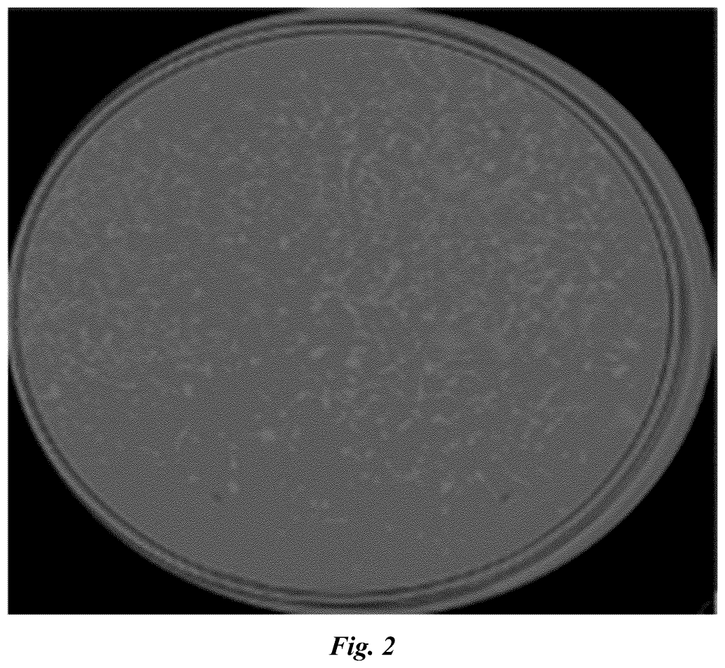

FIG. 2 shows a brightfield image of a cell colony in a well (e.g., of a multi-well plate) with a graphical representation overlay of cell classifications made using a machine-learned model based on the brightfield image. Information from the classification here may be used to classify cell colony health (e.g., using an A-C or A-D metric).

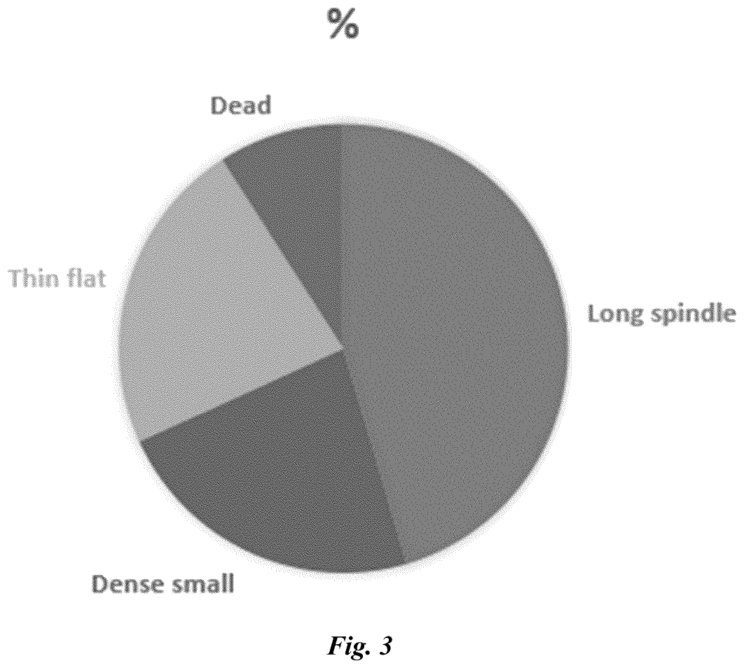

FIG. 3 illustrates a graphical representation of cell classification for cells in a colony, with cells being classified as “dead,” “thin-flat,” “long spindle,” or “dense-small.” Such a classification may be used to classify a cell colony, for example using an A-C or A-D metric. The graphical representation may be derived from output (e.g., a segmentation of a first model of a machine-learned model (e.g., a convolutional neural network, for example having a U-Net architecture) while classification (e.g., grading) of the cell colony using that output may be performed with a second model of a machine-learned model (e.g., a random forest model).

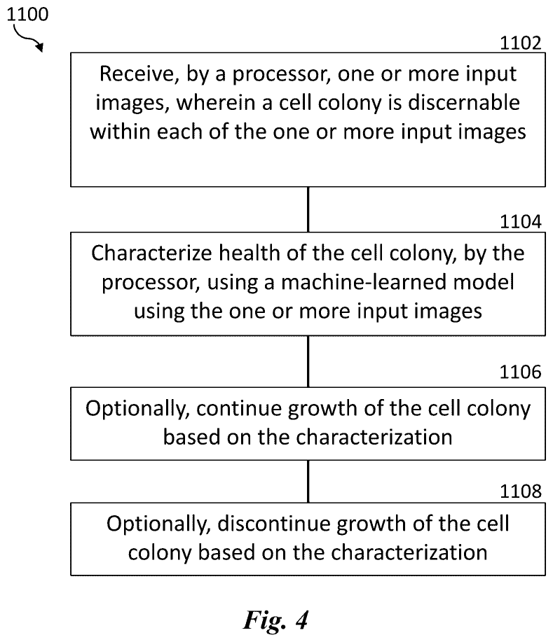

FIG. 4 illustrates an illustrative method 1100 according to certain embodiments of the disclosure. In step 1102, one or more input images in which one or more cell colonies are discernable are received by a processor. In step 1104, the one or more cell colonies are characterized (e.g., classified) (e.g., graded) using a machine-learned model. In optional step 1106, a decision to continue growth of one or more of the one or more cell colonies is made based on the characterization from step 1104 (e.g., based on an A-C or A-D metric for one or more of the one or more cell colonies). For example, a decision to continue to grow a cell colony may be made if a cell colony is sufficiently highly classified on an qualitative metric (e.g., A-C or A-D metric) or if a number and/or percentage of cells or area of a certain classification (e.g., classified as “dense-small”) within a colony exceeds a threshold. Likewise, in optional step 1108, a decision to discard one or more cell colonies is made, for example if one of the aforementioned thresholds is not exceeded. In general, for a given cell colony, at most only one of optional steps 1106 and 1108 will be performed, though both steps may be performed in a method where different colonies are treated differently (e.g., one is allowed to continue growing and another is discarded).

### Exemplary Computing Devices and Components Thereof

Methods of the present disclosure, or portions thereof, may be performed using a processor. The processor may be a part of a computing device and/or computing system.

Systems of the present disclosure may include a processor and/or a memory. The memory may store one or more programs that include instructions that when executed by a processor cause at least a portion of a method disclosed herein to be performed. The system may further include a machine-learned model. Additionally or alternatively, a remotely stored and/or operated machine-learned model may be accessed by a (e.g., the) processor. The processor and/or memory may be a part of a computing device and/or computing system.

One or non-transitory computer readable media may store one or more programs that include instructions that when executed by a (e.g., the) processor cause at least a portion of a method disclosed herein to be performed.

Methods disclosed herein may utilized one or more machine-learned models. A machine-learned model may be or include an artificial neural network. A machine-learned model may employ, for example, a regression-based model (e.g., a logistic regression model), a regularization-based model (e.g., an elastic net model or a ridge regression model), an instance-based model (e.g., a support vector machine or a k-nearest neighbor model), a Bayesian-based model (e.g., a naive-based model or a Gaussian naive-based model), a clustering-based model (e.g., an expectation maximization model), an ensemble-based model (e.g., an adaptive boosting model, a random forest model, a bootstrap-aggregation model, or a gradient boosting machine model), or a neural-network-based model (e.g., a convolutional neural network, a recurrent neural network, autoencoder, a back propagation network, or a stochastic gradient descent network).

In some embodiments, a machine-learned model is or is derived from a decision tree methodology, a neural boosted methodology, a bootstrap forest methodology, a boosted tree methodology, a K nearest neighbors methodology, a generalized regression forward selection methodology, a generalized regression pruned forward selection methodology, a fit stepwise methodology, a generalized regression lasso methodology, a generalized regression elastic net methodology, a generalized regression ridge methodology, a nominal logistic methodology, a support vector machines methodology, a discriminant methodology, a naïve Bayes methodology, or a combination thereof. In some embodiments, a machine-learned model is or is derived from a decision tree methodology, a neural boosted methodology, a bootstrap forest methodology, a boosted tree methodology, a generalized regression lasso methodology, a generalized regression clastic net methodology, a generalized regression ridge methodology, a nominal logistic methodology, a support vector machines methodology, a discriminant methodology, or a combination thereof. In some embodiments, a machine-learned model is or is derived from a decision tree methodology, a neural boosted methodology, a bootstrap forest methodology, a boosted tree methodology, a support vector machines methodology, or a combination thereof.

In embodiments, a machine-learned model has been trained using supervised learning algorithm(s), unsupervised learning algorithm(s), semi-supervised learning algorithm(s) (e.g., partial supervision), weak supervision, transfer, multi-task learning, or any combination thereof. In embodiments, a machine-learned model employs a model that includes parameters (e.g., weights) that are tuned during training of the model. For example, the parameters may be adjusted to minimize a loss function, thereby improving the predictive capacity of the machine learning model. A machine-learned model may be further trained after an initial training period, for example, may be adapted to continuously train as it is used.

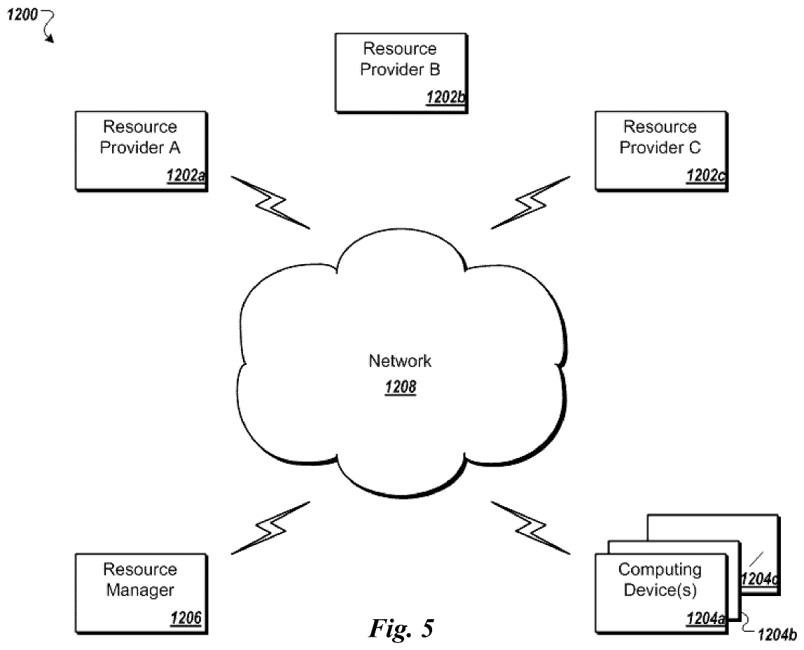

Illustrative embodiments of systems and methods disclosed herein were described above with reference to computations performed locally by a computing device. However, computations performed over a network are also contemplated. FIG. 5 shows an illustrative network environment 1200 for use in the methods and systems described herein. In brief overview, referring now to FIG. 5, a block diagram of an illustrative cloud computing environment 1200 is shown and described. The cloud computing environment 1200 may include one or more resource providers 1202a, 1202b, 1202c (collectively, 1202). Each resource provider 1202 may include computing resources. In some implementations, computing resources may include any hardware and/or software used to process data. For example, computing resources may include hardware and/or software capable of executing algorithms, computer programs, and/or computer applications. In some implementations, illustrative computing resources may include application servers and/or databases with storage and retrieval capabilities. Each resource provider 1202 may be connected to any other resource provider 1202 in the cloud computing environment 1200. In some implementations, the resource providers 1202 may be connected over a computer network 1208. Each resource provider 1202 may be connected to one or more computing device 1204a, 1204b, 1204c (collectively, 1204), over the computer network 1208.

The cloud computing environment 1200 may include a resource manager 1206. The resource manager 1206 may be connected to the resource providers 1202 and the computing devices 1204 over the computer network 1208. In some implementations, the resource manager 1206 may facilitate the provision of computing resources by one or more resource providers 1202 to one or more computing devices 1204. The resource manager 1206 may receive a request for a computing resource from a particular computing device 1204. The resource manager 1206 may identify one or more resource providers 1202 capable of providing the computing resource requested by the computing device 1204. The resource manager 1206 may select a resource provider 1202 to provide the computing resource. The resource manager 1206 may facilitate a connection between the resource provider 1202 and a particular computing device 1204. In some implementations, the resource manager 1206 may establish a connection between a particular resource provider 1202 and a particular computing device 1204. In some implementations, the resource manager 1206 may redirect a particular computing device 1204 to a particular resource provider 1202 with the requested computing resource.

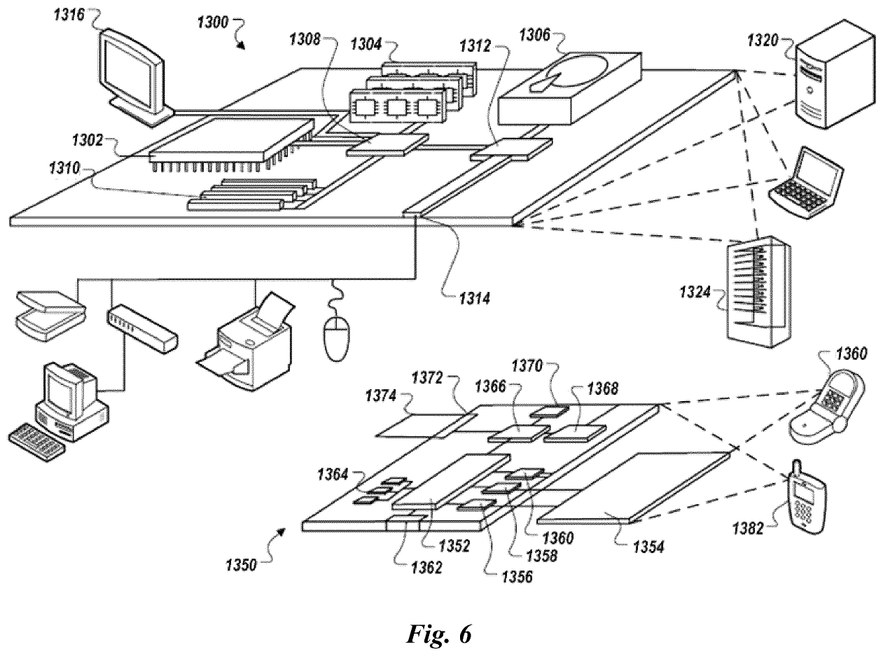

FIG. 6 shows an example of a computing device 1300 and a mobile computing device 1350 that can be used in the methods and systems described in this disclosure. The computing device 1300 is intended to represent various forms of digital computers, such as laptops, desktops, workstations, personal digital assistants, servers, blade servers, mainframes, and other appropriate computers. The mobile computing device 1350 is intended to represent various forms of mobile devices, such as personal digital assistants, cellular telephones, smart-phones, and other similar computing devices. The components shown here, their connections and relationships, and their functions, are meant to be examples only, and are not meant to be limiting.

The computing device 1300 includes a processor 1302, a memory 1304, a storage device 1306, a high-speed interface 1308 connecting to the memory 1304 and multiple high-speed expansion ports 1310, and a low-speed interface 1312 connecting to a low-speed expansion port 1314 and the storage device 1306. Each of the processor 1302, the memory 1304, the storage device 1306, the high-speed interface 1308, the high-speed expansion ports 1310, and the low-speed interface 1312, are interconnected using various busses, and may be mounted on a common motherboard or in other manners as appropriate. The processor 1302 can process instructions for execution within the computing device 1300, including instructions stored in the memory 1304 or on the storage device 1306 to display graphical information for a GUI on an external input/output device, such as a display 1316 coupled to the high-speed interface 1308. In other implementations, multiple processors and/or multiple buses may be used, as appropriate, along with multiple memories and types of memory. Also, multiple computing devices may be connected, with each device providing portions of the necessary operations (e.g., as a server bank, a group of blade servers, or a multi-processor system). Also, multiple computing devices may be connected, with each device providing portions of the necessary operations (e.g., as a server bank, a group of blade servers, or a multi-processor system). Thus, as the term is used herein, where a plurality of functions are described as being performed by “a processor”, this encompasses embodiments wherein the plurality of functions are performed by any number of processors (e.g., one or more processors) of any number of computing devices (e.g., one or more computing devices). Furthermore, where a function is described as being performed by “a processor”, this encompasses embodiments wherein the function is performed by any number of processors (e.g., one or more processors) of any number of computing devices (e.g., one or more computing devices) (e.g., in a distributed computing system).

The memory 1304 stores information within the computing device 1300. In some implementations, the memory 1304 is a volatile memory unit or units. In some implementations, the memory 1304 is a non-volatile memory unit or units. The memory 1304 may also be another form of computer-readable medium, such as a magnetic or optical disk.

The storage device 1306 is capable of providing mass storage for the computing device 1300. In some implementations, the storage device 1306 may be or contain a computer-readable medium, such as a hard disk device, an optical disk device, a flash memory or other similar solid state memory device, or an array of devices, including devices in a storage area network or other configurations. Instructions can be stored in an information carrier. The instructions, when executed by one or more processing devices (for example, processor 1302), perform one or more methods, such as those described above. The instructions can also be stored by one or more storage devices such as computer- or machine-readable mediums (for example, the memory 1304, the storage device 1306, or memory on the processor 1302).

The high-speed interface 1308 manages bandwidth-intensive operations for the computing device 1300, while the low-speed interface 1312 manages lower bandwidth-intensive operations. Such allocation of functions is an example only. In some implementations, the high-speed interface 1308 is coupled to the memory 1304, the display 1316 (e.g., through a graphics processor or accelerator), and to the high-speed expansion ports 1310, which may accept various expansion cards (not shown). In the implementation, the low-speed interface 1312 is coupled to the storage device 1306 and the low-speed expansion port 1314. The low-speed expansion port 1314, which may include various communication ports (e.g., USB, Bluetooth®, Ethernet, wireless Ethernet) may be coupled to one or more input/output devices, such as a keyboard, a pointing device, a scanner, or a networking device such as a switch or router, e.g., through a network adapter.

The computing device 1300 may be implemented in a number of different forms, as shown in the figure. For example, it may be implemented as a standard server 1320, or multiple times in a group of such servers. In addition, it may be implemented in a personal computer such as a laptop computer 1322. It may also be implemented as part of a rack server system 1324. Alternatively, components from the computing device 1300 may be combined with other components in a mobile device (not shown), such as a mobile computing device 1350. Each of such devices may contain one or more of the computing device 1300 and the mobile computing device 1350, and an entire system may be made up of multiple computing devices communicating with each other.

The mobile computing device 1350 includes a processor 1352, a memory 1364, an input/output device such as a display 1354, a communication interface 1366, and a transceiver 1368, among other components. The mobile computing device 1350 may also be provided with a storage device, such as a micro-drive or other device, to provide additional storage. Each of the processor 1352, the memory 1364, the display 1354, the communication interface 1366, and the transceiver 1368, are interconnected using various buses, and several of the components may be mounted on a common motherboard or in other manners as appropriate.

The processor 1352 can execute instructions within the mobile computing device 1350, including instructions stored in the memory 1364. The processor 1352 may be implemented as a chipset of chips that include separate and multiple analog and digital processors. The processor 1352 may provide, for example, for coordination of the other components of the mobile computing device 1350, such as control of user interfaces, applications run by the mobile computing device 1350, and wireless communication by the mobile computing device 1350.

The processor 1352 may communicate with a user through a control interface 1358 and a display interface 1356 coupled to the display 1354. The display 1354 may be, for example, a TFT (Thin-Film-Transistor Liquid Crystal Display) display or an OLED (Organic Light Emitting Diode) display, or other appropriate display technology. The display interface 1356 may comprise appropriate circuitry for driving the display 1354 to present graphical and other information to a user. The control interface 1358 may receive commands from a user and convert them for submission to the processor 1352. In addition, an external interface 1362 may provide communication with the processor 1352, so as to enable near area communication of the mobile computing device 1350 with other devices. The external interface 1362 may provide, for example, for wired communication in some implementations, or for wireless communication in other implementations, and multiple interfaces may also be used.

The memory 1364 stores information within the mobile computing device 1350. The memory 1364 can be implemented as one or more of a computer-readable medium or media, a volatile memory unit or units, or a non-volatile memory unit or units. An expansion memory 1374 may also be provided and connected to the mobile computing device 1350 through an expansion interface 1372, which may include, for example, a SIMM (Single In Line Memory Module) card interface. The expansion memory 1374 may provide extra storage space for the mobile computing device 1350, or may also store applications or other information for the mobile computing device 1350. Specifically, the expansion memory 1374 may include instructions to carry out or supplement the processes described above, and may include secure information also. Thus, for example, the expansion memory 1374 may be provided as a security module for the mobile computing device 1350, and may be programmed with instructions that permit secure use of the mobile computing device 1350. In addition, secure applications may be provided via the SIMM cards, along with additional information, such as placing identifying information on the SIMM card in a non-hackable manner.

The memory may include, for example, flash memory and/or NVRAM memory (non-volatile random access memory), as discussed below. In some implementations, instructions are stored in an information carrier and, when executed by one or more processing devices (for example, processor 1352), perform one or more methods, such as those described above. The instructions can also be stored by one or more storage devices, such as one or more computer- or machine-readable mediums (for example, the memory 1364, the expansion memory 1374, or memory on the processor 1352). In some implementations, the instructions can be received in a propagated signal, for example, over the transceiver 1368 or the external interface 1362.

The mobile computing device 1350 may communicate wirelessly through the communication interface 1366, which may include digital signal processing circuitry where necessary. The communication interface 1366 may provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication may occur, for example, through the transceiver 1368 using a radio-frequency. In addition, short-range communication may occur, such as using a Bluetooth®, Wi-Fi™, or other such transceiver (not shown). In addition, a GPS (Global Positioning System) receiver module 1370 may provide additional navigation- and location-related wireless data to the mobile computing device 1350, which may be used as appropriate by applications running on the mobile computing device 1350.

The mobile computing device 1350 may also communicate audibly using an audio codec 1360, which may receive spoken information from a user and convert it to usable digital information. The audio codec 1360 may likewise generate audible sound for a user, such as through a speaker, e.g., in a handset of the mobile computing device 1350. Such sound may include sound from voice telephone calls, may include recorded sound (e.g., voice messages, music files, etc.) and may also include sound generated by applications operating on the mobile computing device 1350.

The mobile computing device 1350 may be implemented in a number of different forms, as shown in the figure. For example, it may be implemented as a cellular telephone 1380. It may also be implemented as part of a smart-phone 1382, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

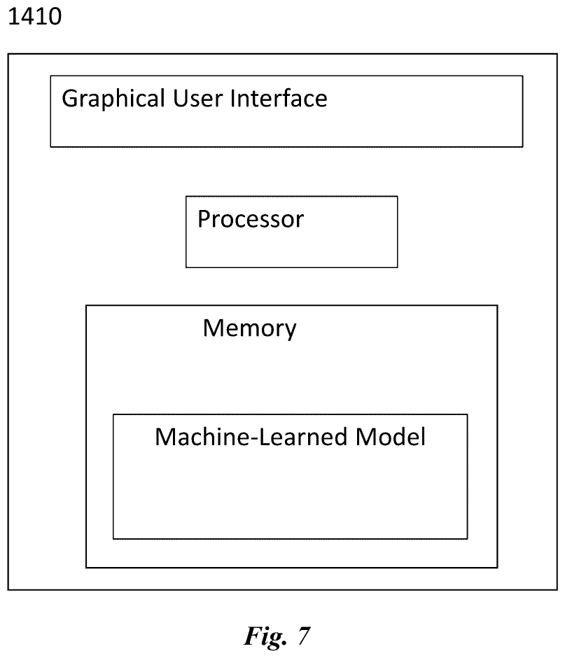

FIG. 7 illustrates an example system 1410 that can perform at least a portion of methods disclosed herein. The system 1410 includes one or more processors, a memory storing one or more programs that include instructions that are executable by the processor(s), optionally a machine learning module stored in the memory, and optionally a graphical user interface.

Details of exemplary image acquisition processes that may be used in systems and methods disclosed herein are described in U.S. Pat. Nos. 9,600,710 and 11,282,175 and U.S. Patent Publication Nos. 2022/0406080 and 2022/0404258, the disclosure of each of which is hereby incorporated by reference herein in its entirety.

Methods and systems of growing cell cultures or colonies, for example from a single seed cell, for example in a well of a well-late, which may be used in methods and systems disclosed herein, are described in U.S. Pat. No. 11,279,910, U.S. Patent Publication Nos. 2022/0193675 and 2021/0163875, and British Patent Publication No. GB2603922A, the disclosure of each of which is hereby incorporated by reference herein in its entirety.

It is contemplated that systems, devices, methods, and processes of the disclosure encompass variations and adaptations developed using information from the embodiments described herein. Adaptation and/or modification of the systems, devices, methods, and processes described herein may be performed by those of ordinary skill in the relevant art.

Throughout the description, where articles, devices, and systems are described as having, including, or comprising specific components, or where processes and methods are described as having, including, or comprising specific steps, it is contemplated that, additionally, there are articles, devices, and systems according to certain embodiments of the present disclosure that consist essentially of, or consist of, the recited components, and that there are processes and methods according to certain embodiments of the present disclosure that consist essentially of, or consist of, the recited processing steps.

It should be understood that the order of steps or order for performing certain action is immaterial so long as operability is not lost. Moreover, two or more steps or actions may be conducted simultaneously. As is understood by those skilled in the art, the terms “over”, “under”, “above”, “below”, “beneath”, and “on” are relative terms and can be interchanged in reference to different orientations of the layers, elements, and substrates included in the present disclosure. For example, a first layer on a second layer, in some embodiments means a first layer directly on and in contact with a second layer. In other embodiments, a first layer on a second layer can include another layer there between.

Headers have been provided for the convenience of the reader and are not intended to be limiting with respect to the claimed subject matter.

In this application, unless otherwise clear from context or otherwise explicitly stated, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) where ranges are provided, endpoints are included.

Various sets of exemplary numbered embodiments have been described above. Those of ordinary skill in the art will readily appreciate that embodiments or one or more features thereof from different ones of the sets can be interchanged and/or combined.

Certain embodiments of the present disclosure were described above. It is, however, expressly noted that the present disclosure is not limited to those embodiments, but rather the intention is that additions and modifications to what was expressly described in the present disclosure are also included within the scope of the disclosure. Moreover, it is to be understood that the features of the various embodiments described in the present disclosure were not mutually exclusive and can exist in various combinations and permutations, even if such combinations or permutations were not made express, without departing from the spirit and scope of the disclosure. The disclosure has been described in detail with particular reference to certain embodiments thereof, but it will be understood that variations and modifications can be effected within the spirit and scope of the claimed invention.

Various embodiments of the present invention useful for, inter alia, characterizing (e.g., classifying) cell [e.g., stem cell (e.g., mesenchymal stem cell)] colony health may be characterized by the embodiments following this paragraph (and before the actual claims provided at the end of this application). These embodiments form a part of the written description of this application. Accordingly, subject matter of the following embodiments may be claimed in later proceedings involving this application or any application claiming priority based on this application. Inclusion of such embodiments should not be construed to mean that the actual claims do not cover the subject matter of the embodiments. Thus, a decision not to present these embodiments in later proceedings should not be construed as a donation of the subject matter to the public. Without limitation, various embodiments of the present invention (prefaced with the letter “E” so as to avoid confusion with the actual claims of this application) include:

E1. A method (e.g., a computer-implemented method) of characterizing (e.g., classifying) cell [e.g., stem cell (e.g., mesenchymal stem cell)] colony health, the method comprising:
receiving, by a processor (e.g., of a computing device), one or more input images, wherein a cell colony is discernable within each of the one or more input images; and
characterizing (e.g., classifying) (e.g., grading) health of the cell colony, by the processor, using a machine-learned model using the one or more input images as input to the model.

E2. The method of embodiment E1, wherein the colony is a mesenchymal stem cell colony.

E3. The method of embodiment E1 or embodiment E2, wherein the machine-learned model classifies health of the cell colony based on cell morphology and/or colony morphology.

E4. The method of any one of embodiments E1-3, wherein the machine-learned model classifies health of the cell colony further based at least on one or more intracellular features.

E5. The method of any one of embodiments E1-4, wherein the machine-learned model comprises a first model and a second model and the one or more input images are input for the first model and input for the second model is based on output from the first model and output from the second model characterizes the one or more cell colonies.

E6. The method of embodiment E5, wherein the output from the second model is a grade [e.g., an A-C or A-D grade (e.g., an A-C or A-D metric (e.g., where “A” represents a colony comprising mostly dense small cells, “B” represents a colony comprising mostly dense small cells and some long spindle cells, and “C” represents a colony comprising a high amount of dead cells, some long spindle cells, and few dense small cells)].

E7. The method of embodiment E5 or embodiment E6, wherein the output from the first model is a segmentation of the one or more images (e.g., into a plurality of qualitative classifications).

E8. The method of any one of embodiments E5-7, comprising determining a ratio of classes (e.g., percentage of image area classified into the classes) from the output from the first model (e.g., wherein the output from the first model is the ratio of the classes).

E9. The method of embodiment E8, wherein the input to the second model is based on the ratio of the classes.

E10. The method of any one of embodiments E5-9, comprising determining an area fraction of each of a plurality of classes (e.g., percentage of image area classified into the classes) from the output from the first model (e.g., wherein the output from the first model is the relative area of the classes).

E11. The method of embodiment E10, wherein the input to the second model is based on the area fraction of the classes.

E12. The method of any one of embodiments E8-11, wherein the classes correspond to cell morphology and/or colony morphology.

E13. The method of any one of embodiments E5-12, wherein the first model is a convolutional neural network and the one or more input images are input for the convolutional neural network.

E14. The method of embodiment E13, wherein the convolutional neural network has a U-Net architecture (e.g., a modified U-Net architecture).

E15. The method of embodiment E13 or embodiment E14, wherein the characterizing comprises segmenting the one or more input images with the convolutional neural network.

E16. The method of embodiment E15, wherein the segmenting comprises segmenting the one or more input images into classes that correspond to cell morphology and/or colony morphology [e.g., one or more qualitative classifications for cells in each of the one or more colonies (e.g., “long-spindle cells,” “thin-flat cells,” “dead” (or “dead-cell”), or “dense-small”)].

E17. The method of any one of embodiments E5-16, wherein the second model is a decision-tree-based model.

E18. The method of any one of embodiments E5-17, wherein the second model is an ensemble model.

E19. The method of any one of embodiments E5-18, wherein the second model is a random forest model.

E20. The method of any one of embodiments E1-19, wherein the one or more input images comprises an input image (e.g., a single input image) for each of a plurality of wells in a multi-well plate (e.g., each well in the multi-well plate) (e.g., wherein the images are input separately into the machine-learned model).

E21. The method of any one of embodiments E1-20, wherein the one or more colonies comprises a colony (e.g., a single colony) for each of a plurality of wells in a multi-well plate.

E22. The method of any one of embodiments E1-21, wherein the one or more input images are one or more multi-frame images (e.g., corresponding to different z-stack image planes).

E23. The method of embodiment E22, wherein the multi-frame images are 2-frame images or 3-frame images (e.g., 2-frame whole-well images or 3-frame whole-well images).

E24. The method of embodiment E22 or embodiment 23, wherein each frame of each of the one or more multi-frame images is input to the machine-learned model (e.g., a first model thereof) in a separate input channel.

E25. The method of any one of embodiments E1-24, wherein the characterizing comprises outputting from the machine-learned model one of a plurality of classifications for each of the one or more colonies, wherein the plurality of classifications comprises three or more distinct classifications [e.g., four or more (e.g., four), five or more (e.g., five), six or more (e.g., six) distinct classifications].

E26. The method of any one of embodiments E1-25, wherein the characterizing comprises determining (e.g., outputting from the machine-learned model) one or more qualitative classifications for the one or more colonies (e.g., a collective classification for the one or more colonies collectively) [e.g., according to a qualitative metric (e.g., based on colony perimeter morphology and/or cell-level classification in the cell colony) (e.g., an A-C or A-D metric (e.g., where “A” represents a colony comprising mostly dense small cells, “B” represents a colony comprising mostly dense small cells and some long spindle cells, and “C” represents a colony comprising a high amount of dead cells, some long spindle cells, and few dense small cells))].

E27. The method of any one of embodiments E1-26, wherein the characterizing comprises determining (e.g., outputting from the machine-learned model) one or more qualitative classifications for cells in each of the one or more colonies [e.g., long-spindle cell, thin-flat cell, dead, and/or dense-small cell (e.g., wherein a colony is or may be classified as comprising one or more dead cells or an amount of dead cells above a threshold, optionally in addition to another classification)] [e.g., wherein the qualitative classification for the cells is used (e.g., by the machine-learned model) to determine one or more (e.g., qualitative) classifications for the one or more colonies (e.g., an A-D metric)].

E28. The method of any one of embodiments E1-27, comprising outputting, by the processor, (e.g., from the machine-learned model) a graphical representation of quality of the one or more cell colonies (e.g., a pie chart) (e.g., and saving and/or displaying, by the processor, the graphical representation) (e.g., wherein the characterizing comprises the outputting).

E29. The method of any one of embodiments E1-28, comprising ranking (e.g., by the processor) the one or more cell colonies based on the characterizing (e.g., comprising outputting from the machine-learned model a ranking of the one or more cell colonies).

E30. The method of any one of embodiments E1-29, wherein the one or more cell colonies is two or more cell colonies (e.g., at least three cell colonies, at least four cell colonies) (e.g., is a number of cell colonies corresponding to a number of wells in a multi-well plate).

E31. The method of any one of embodiments E1-30, wherein each of the one or more cell colonies is contained within a well (e.g., a single well) of a multi-well plate.

E32. The method of any one of embodiments E1-31, wherein the one or more cell colonies is a plurality of cell colonies and at least one (e.g., only one) of (e.g., each of) the one or more cell colonies is contained in each of a plurality of wells of a multi-well plate.

E33. The method of any one of embodiments E1-32, wherein the one or more input images correspond to the one or more cell colonies within no more than 14 days (e.g., within 12 days, within 10 days, within 8 days, within 6 days, or within 4 days) (e.g., and after at least 1 day or at least 2 days) of beginning to grow (e.g., culture) the one or more cell colonies.

E34. The method of any one of embodiments E1-33, comprising continuing to grow one or more of the one or more cell colonies based on the characterizing the health of the one or more cell colonies (e.g., based on a grade output from the machine-learned model).

E35. The method of embodiment E34, wherein it is determined to continue to grow the one or more of the one or more cell colonies within no more than 14 days (e.g., within 12 days, within 10 days, within 8 days, within 6 days, or within 4 days) (e.g., and after at least 1 day or at least 2 days) of when culturing of the one or more of the one or more cell colonies began.

E36. The method of any one of embodiments E1-35, comprising discarding one or more of the cell colonies based on the characterizing the health of the one or more cell colonies.

E37. The method of embodiment E36, wherein the discarding occurs within no more than 14 days (e.g., within 12 days, within 10 days, within 8 days, within 6 days, or within 4 days) (e.g., and after at least 1 day or at least 2 days) of when culturing of the one or more of the one or more cell colonies began.

E38. The method of any one of embodiments E1-37, wherein each of the one or more input images is a stitched image that has been assembled from a set of constituent images (e.g., at least 10, at least 100, or at least 1000 constituent images).

E39. The method of any one of embodiments E1-38, wherein the characterizing using the machine-learned model comprises inputting, by the processor, one or more timepoints corresponding to the one or more input images into the machine-learned model (e.g., using metadata from the one or more input images).

E40. The method of any one of embodiments E1-39, wherein the one or more input images are brightfield images (e.g., whole-well brightfield images) (e.g., 2-frame whole-well brightfield images).

E41. The method of any one of embodiments E1-40, wherein the one or more input images are images of the one or more cell colonies in an unstained and/or undyed state.

E42. The method of any one of embodiments E1-41, wherein the one or more input images are label free.

E43. The method of any one of embodiments E1-42, wherein the one or more input images correspond to one or more whole wells (e.g., in a multi-well plate) [e.g., each of the one or more input images corresponds to multiple wells in a multi-well plate (e.g., each of the one or more input images corresponds to each well of the multi-well plate)].

E44. The method of any one of embodiments E1-43, wherein the characterizing the one or more cell colonies using the machine-learned model is based on a morphology and/or size of the one or more cell colonies within the images

E45. The method of any one of embodiments E1-44, wherein the machine-learned model has been trained using one or more datasets of images that have been annotated based on cell colony class.

E46. The method of embodiment E45, wherein the cell class is a qualitative annotation (e.g., corresponding to a long-spindle cell, thin-flat cell, dead, and/or dense-small cell classification).

E47. The method of embodiment E45 or embodiment E46, wherein the annotations are manual annotations.

E48. The method of any one of embodiments E45-47, wherein the one or more datasets comprise brightfield images.

E49. The method of any one of embodiments E45-48, wherein the one or more datasets comprise fluorescence images.

E50. The method of any one of embodiments E1-49, wherein the one or more input images have been preprocessed (e.g., normalized and/or binned down).

E51. The method of any one of embodiments E1-50, wherein the method is performed as part of a cell line development process.

E52. The method of any one of embodiments E1-51, wherein the method is performed as part of a pluripotency-based assay.

E53. The method of any one of embodiments E1-52, wherein the machine-learned model comprises a convolutional neural network.

E54. The method of embodiment E53, wherein the convolutional neural network has a U-Net architecture (e.g., a modified U-Net architecture).

E55. The method of any one of embodiments E1-53, wherein the characterizing comprises segmenting the one or more input images with the machine-learned model.

E56. The method of embodiment E55, wherein an output of the machine-learned model is a segmentation of the one or more input images (e.g., as a mask or an area fraction of classes in a classification scheme).

E57. A system comprising the processor; a memory; and one or more programs [e.g., and the machine-learned model (e.g., stored in the memory)], wherein the one or more programs are stored in the memory and are executable by the processor, the one or more programs comprising instructions for implementing at least a portion of the method (e.g., the method) according to any one of embodiments E1-56.

E58. One or more non-transitory computer readable storage media comprising one or more programs comprising instructions for implementing at least a portion of the method (e.g., the method) according to any one of embodiments E1-56.

The embodiments of the invention described above are intended to be merely exemplary; numerous variations and modifications will be apparent to those skilled in the art. All such variations and modifications are intended to be within the scope of the present invention as defined in any appended claims.

What is claimed is:

1. A method of characterizing cell colony health, the method comprising:
receiving, by a processor, one or more input images, wherein a cell colony is discernable within each of the one or more input images; and
characterizing health of the cell colony, by the processor, using a machine-learned model using the one or more input images as input to the model.

2. The method of claim 1, wherein the colony is a mesenchymal stem cell colony.

3. The method of claim 1, wherein the machine-learned model classifies health of the cell colony based on cell morphology and/or colony morphology.

4. The method of claim 1, wherein the machine-learned model classifies health of the cell colony further based at least on one or more intracellular features.

5. The method of claim 1, wherein the machine-learned model comprises a first model and a second model and the one or more input images are input for the first model and input for the second model is based on output from the first model and output from the second model characterizes the one or more cell colonies.

6. The method of claim 5, wherein the output from the second model is a grade.

7. The method of claim 1, wherein the characterizing comprises outputting from the machine-learned model one of a plurality of classifications for each of the one or more colonies, wherein the plurality of classifications comprises three or more distinct classifications.

8. The method of claim 1, wherein the characterizing comprises determining one or more qualitative classifications for the one or more colonies.

9. The method of claim 1, wherein the characterizing comprises determining one or more qualitative classifications for cells in each of the one or more colonies.

10. The method of claim 1, comprising ranking the one or more cell colonies based on the characterizing.

11. The method of claim 1, wherein the one or more input images correspond to the one or more cell colonies within no more than 14 days of beginning to grow the one or more cell colonies.

12. The method of claim 1, comprising continuing to grow one or more of the one or more cell colonies based on the characterizing the health of the one or more cell colonies.

13. The method of claim 1, comprising discarding one or more of the cell colonies based on the characterizing the health of the one or more cell colonies.

14. The method of claim 13, wherein the discarding occurs within no more than 14 days of when culturing of the one or more of the one or more cell colonies began.

15. The method of claim 1, wherein each of the one or more input images is a stitched image that has been assembled from a set of constituent images.

16. The method of claim 1, wherein the characterizing using the machine-learned model comprises inputting, by the processor, one or more timepoints corresponding to the one or more input images into the machine-learned model.

17. The method of claim 1, wherein the characterizing the one or more cell colonies using the machine-learned model is based on a morphology and/or size of the one or more cell colonies within the images

18. The method of claim 1, wherein the machine-learned model has been trained using one or more datasets of images that have been annotated based on cell colony class.

19. A system comprising the processor; a memory; and one or more programs, wherein the one or more programs are stored in the memory and are executable by the processor, the one or more programs comprising instructions for implementing at least a portion of the method of claim 1.

20. One or more non-transitory computer readable storage media comprising one or more programs comprising instructions for implementing at least a portion of the method of claim 1.
